(19) United States
(12) Patent Application Publication
(10) Pub. No.: US 2010/0125188 A1
(43) Pub. Date: May 20, 2010

(54) MOTION CORRELATED PULSE OXIMETRY

(75) Inventors: Josh D. Schilling, Eden Praire, MN (US); Kenneth W. Thomas, Blaine, MN (US)

Correspondence Address: SCHWEGMAN, LUNDBERG & WOESSNER, P.A., P.O. BOX 2938, MINNEAPOLIS, MN, 55402, US

(73) Assignee: Nonin Medical, Inc., Plymouth, MN (US)

(21) Appl. No.: 12/272,979

(22) Filed: Nov. 18, 2008

(51) Int. Cl. A61B 5/1455; G08C 19/16

(52) U.S. Cl. 600/336; 340/870.01

(57) ABSTRACT

A device includes a first sensor, a motion sensor, and a processor. The first sensor has an optical detector and an optical emitter. The optical detector generates a first output using the optical emitter. The first output corresponds to a physiological parameter of a user. The motion sensor generates a motion output corresponding to a detected motion of the user. The motion sensor is configured for attachment to the user. The processor is coupled to the first sensor by a first link and coupled to the motion sensor by a second link. At least one of the first link and the second link includes a wireless communication channel. The processor generates a processor output using the first output and the motion output.

### BACKGROUND

Blood oxygenation can be determined using pulse oximetry. In some environments, pulse oximetry accuracy is insufficient to allow proper treatment or diagnosis of a patient. Current technology for pulse oximetry is inadequate.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 includes a block diagram of a system according to one example.

FIG. 2 includes a pictorial representation of a system according to one example.

FIG. 3 includes a flow chart of a method according to one example.

FIG. 4 includes a motion sensor with a coordinate system.

### DETAILED DESCRIPTION

By way of overview, an example of the present subject matter includes a motion compensated physiological sensor. In one example, the physiological sensor includes a pulse oximetry sensor. Motion detected by the motion sensor can be used to compensate or correct the pulse oximetry data provided by the pulse oximetry sensor. In one example, motion detected by the motion sensor is used to generate a notification. The notification can be a signal provided to the user, a physician, or other caregiver or the notification can be stored in a memory or other storage device.

In one example, the motion sensor is configured to be worn by the user. For example, the motion sensor can include an accelerometer. The accelerometer can have one or more axes of sensitivity. The accelerometer can be attached to a selected body portion of the user. For example, a torso-worn accelerometer can be used in a sleep study or used to detect vibrations or movement of a user during transit from one location to another. As another example, a wrist-worn or ankle-worn accelerometer can detect limb movement of the user. Movement artifacts detected during a sleep study, for example, can be correlated to measured oximetry or pulse data.

In one example, the present subject matter includes a body worn pulse oximetry sensor that is coupled by a wired connection to a body worn accelerometer.

The sensor can be configured to detect pulse oximetry using an optical detector coupled to a finger, a toe, an ear lobe, a forehead, or other tissue. The sensor can be configured for long term monitoring or short term monitoring.

In addition to a pulse oximetry sensor, other types of physiological sensors are also contemplated. For example, the sensor can include a sensor configured to measure pulse rate, measure oxygen saturation, or arterial hemoglobin.

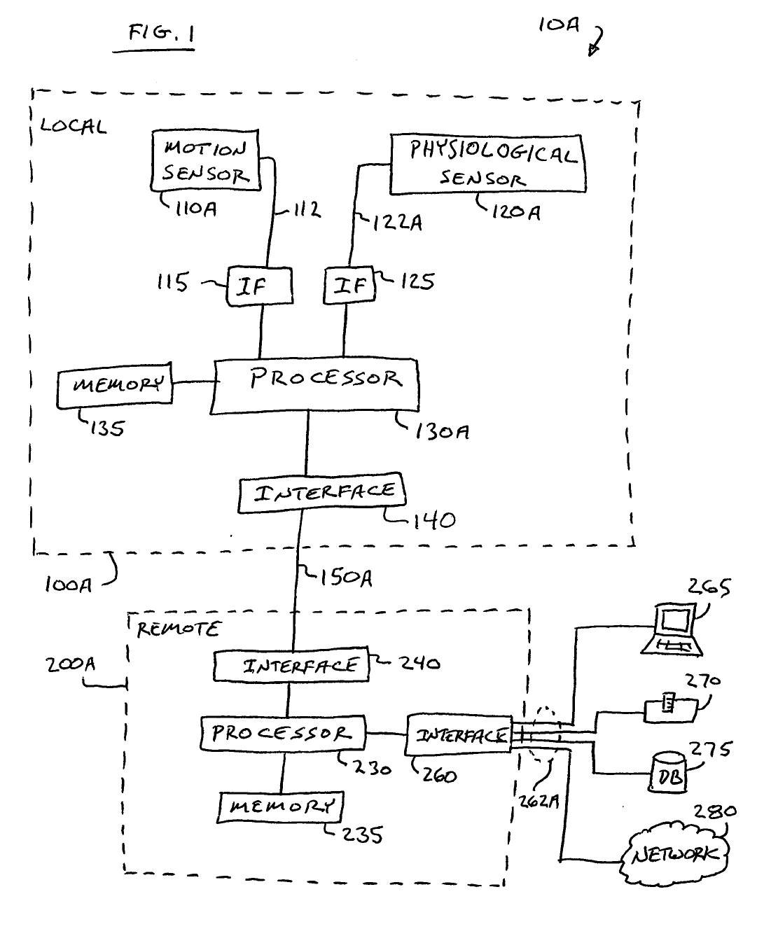

FIG. 1 includes a block diagram of system 10A according to one example. In the example shown in the figure, system 10A includes local unit 100A coupled by link 150A to remote unit 200A.

Local unit 100A includes motion sensor 110A. Motion sensor 110A can include an accelerometer or other device for detecting acceleration or motion. Motion sensor 110A can be sensitive to motion along a single-axis or along multiple axes. Motion sensor 110A provides an electrical output signal corresponding to a detected magnitude and direction of acceleration.

Local unit 100A includes physiological sensor 120A. Physiological sensor 120A can include a pulse oximetry sensor having a light emitter (source) and having a light detector. A pulse oximetry sensor provides an electrical output signal corresponding to a measure of blood oxygenation. According to one example, blood oxygenation is based on modulation of light detected by the light detector.

An output from motion sensor 10A is provided to processor 130A by link 112 and an output from physiological sensor 120A is provided to processor 130A by link 122A. Link 112 can be wired or wireless and in the example shown, includes interface 115. In a similar manner, link 122A can be wired or wireless and in the example shown, includes interface 125.

An example of a wired link can include a copper conductor. Examples of a wireless link can include an optical communication link or a radio frequency communication link. According to one example, a radio frequency communication link can include a Bluetooth communication link. Bluetooth is a wireless protocol utilizing short-range communications technology.

Interface 115 or interface 125 can include a radio frequency transceiver or other telemetry unit. In one example, interface 115 or interface 125 includes a driver, an analog-to-digital (ADC) converter, or other circuitry to interface processor 130A to motion sensor 110A and physiological sensor 120A.

Link 112, interface 115, link 122A, or interface 125 can be unidirectional or bidirectional. In other words, processor 130A can both receive and transmit data between either one or both of motion sensor 110A and physiological sensor 120A.

Processor 130A can include a digital data processor (such as a central processing unit or a microprocessor), an analog processor, or a mixed signal processor. In the example shown, processor 130A is coupled to memory 135. Memory 135 can provide storage for instructions to control operation of processor 130A. Memory 135 can provide data storage for processor 130A.

Processor 130A of local unit 100A communicates with processor 230 of remote unit 200A using link 150A. Link 150A can be wired or wireless. Processor 130A is coupled to link 150A by interface 140. Interface 140 can include a transceiver, a driver, or other circuit to communicate using link 150A. In one example, interface 140 includes an electrical connector.

In the example shown, remote unit 200A includes interface 240, processor 230, memory 235, and interface 260.

Interface 240, like interface 140, can include a transceiver or other circuit to provide or receive a signal using link 150A. Processor 230 can include a digital data processor, an analog processor, or a mixed signal processor, and in the example shown, can execute instructions stored using memory 235. Interface 260 is coupled to output port 262 which provides a coupling to externalities such as computer 265, printer 270, database 275, and network 280.

Motion detected by motion sensor 110A can be used to correlate or compensate the data generated by physiological sensor 120A. Various algorithms or techniques can be implemented using any of processor 130A, processor 230A, or other processor (such as a processor of computer 265). For example, processor 130A can be configured to execute instructions to generate a processor output based on a signal received from motion sensor 110A and physiological sensor 120A. The instructions can use the detected motion to adjust weighting of the data from the physiological sensor 120A. In one example, motion data is used to subtract or nullify portions of the data generated by physiological sensor 120A. In one example, a processor executes instructions to compensate for periodic movement arising from ambulance travel or other motion.

Interface 260 can include a wireless transceiver. For example, interface 260 can include a radio frequency transceiver (such as a Bluetooth transceiver) to allow wireless telemetry to a remote computer.

In the example shown, computer 265 has a display and can include a desktop or laptop computer or other processor. Printer 270 can include a laser printer. Database 275 can include, for example, a storage device or other structure to store data corresponding to motion and physiological parameters of the user. Network 280 can include a local area network (LAN) such as an Ethernet or a wide area network (WAN) such as the internet.

Local unit 100A can include a battery or other power supply. Remote unit 200A can include a battery or other power supply.

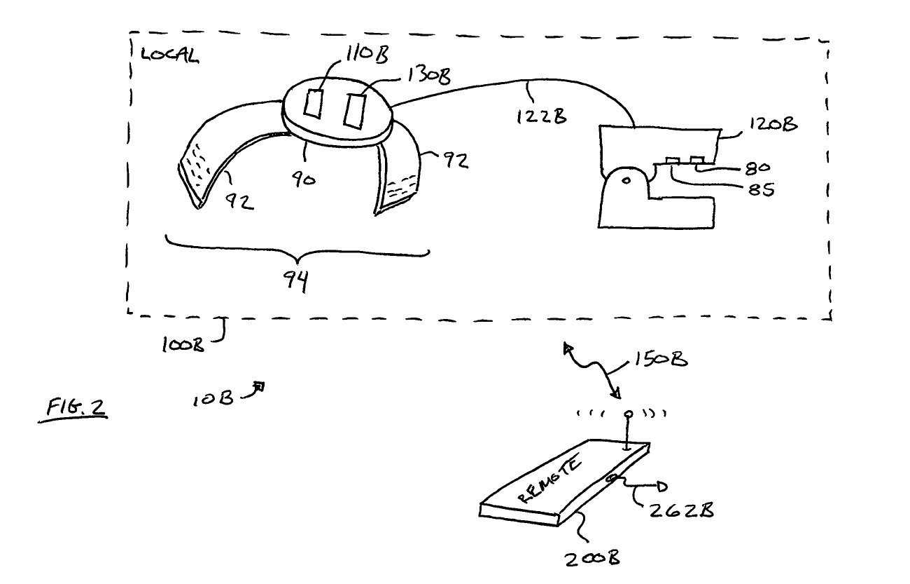

FIG. 2 includes a pictorial representation of system 10B according to one example. In the example shown, system 10B includes local unit 100B and remote unit 200B. Local unit 100B includes physiological sensor 120B, and in the example shown, sensor 120B includes a pulse oximetry sensor configured for use on a finger of a user. A pulse oximetry sensor as shown in the figure includes optical emitter 80 and optical detector 85. An output signal from optical detector 85 corresponds to the blood oxygenation of the user at the sensor site. In one example, local unit 100B includes a battery power supply as part of one or both of device 94 and sensor 120B. Local unit 100B is configured for lightweight, portable use and affords mobility for the user.

The output signal from physiological sensor 120B is communicated by link 122B to device 94. Link 122B can include a wired or wireless communication channel. Device 94, in the example shown, is configured for wearing on a wrist or ankle of the user. Device 94 includes straps 92 configured to encircle and to hold housing 90 in close contact with the user. Sensor 110B is affixed to housing 90 and includes an accelerometer. Sensor 110B can be sensitive to motion along one axis or multiple axes (such as two, three, or more). Housing 90 also includes processor 130B. In one example, processor 130B includes a digital processor to generate a processor output using a signal detected by physiological sensor 120B and motion sensor 110B. In various examples, device 94 includes a display and user-operable controls.

Housing 90 also includes other circuitry such as interface 115, interface 125, interface 140, and memory 135. In one example, housing 90 includes a transceiver configured to communicate wirelessly with remote unit 200B.

Remote unit 200B, in the example shown, includes an antenna to communicate wirelessly with local unit 100B via link 150B. In addition, remote unit 200B includes a connector for coupling, via port 262B, with externalities.

System 10A, as shown in FIG. 1, depicts a general view in which local unit 100A includes motion sensor 110A, physiological sensor 120A, and processor 130A. System 10A can be configured in various combinations of one, two, or three housings with separate housings coupled by various communication channels. A housing can be fabricated of plastic, metal, or other material.

For example, FIG. 2 illustrates system 10B in which a first housing includes physiological sensor 120B and a second housing includes motion sensor 110B and processor 130B. The first housing and the second housing communicate using link 122B. Motion sensor 10B can be a micromachined or nanofabricated device and mounted on a printed wire board (PWB) or other substrate along with processor 130B or other elements.

In one example, motion sensor 110A is integrated in a first housing and a second housing includes processor 130A and physiological sensor 120A. For example, processor 130A and optical elements of physiological sensor 120A can be affixed to a flexible circuit substrate. The substrate can include an aperture for an optical element of a pulse oximetry sensor.

In one example, a first housing include motion sensor 110A and physiological sensor 120A and a second housing includes processor 130A.

In one example, a first housing includes motion sensor 110A, a second housing includes physiological sensor 120A, and a third housing includes processor 130A, and the various housings are in communication with wired communication links or wireless communication links. In one example, a wired communication link includes an electrical connector such as a zero-insertion force (ZIF) connector. Examples of a wireless communication link include a radio frequency transceiver and an optical communication system (such as fiber optic bundle).

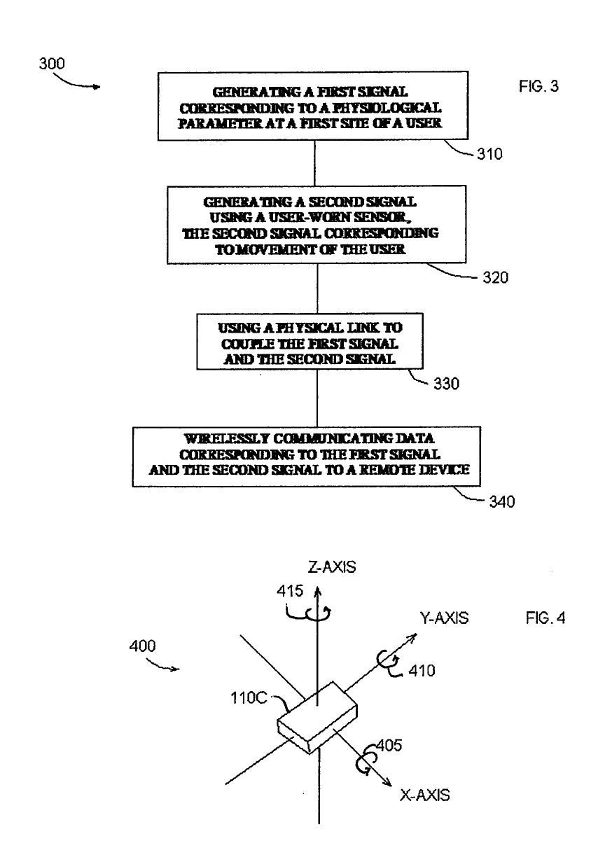

FIG. 3 includes a flow chart of method 300 according to one example. At 310, method 300 includes generating a first signal corresponding to a physiological parameter at a first site of a user. For example, the physiological parameter can correspond to blood oxygenation as measured by a pulse oximetry sensor coupled to a user. The sensor can be affixed to a toe, a finger, an ear lobe, or other tissue of a user. In one example, the physiological parameter can correspond to tissue oxygenation as measured by a suitable sensor coupled to a user.

At 320, method 300 includes generating a second signal using a user-worn sensor, the second signal corresponding to movement of the user. The second signal can correspond to movement of a portion of the user that differs from that of the site used for measuring the physiological parameter. For example, the physiological parameter can be derived from a toe measurement and the user movement can correspond to motion of the user's arm. The first signal and the second signal can correspond to the same portion of the user, such as a torso.

At 330, method 300 includes using a communication link to couple the first signal and the second signal. The communication link, in one example, includes a physical link such as a wired connection or an optical fiber.

At 340, method 300 includes wirelessly communicating data corresponding to the first signal and the second signal to a remote device. The data can be wirelessly communicated using, for example, a radio frequency transceiver, an optical coupling or other means.

A processor executing instructions can be used to receive the data and identify motion artifacts in the data from a physiological sensor. A motion artifact can be classified according to magnitude, direction, or other parameter. In addition, a motion artifact can be correlated with the data from the physiological sensor. Correlating can include classifying data according to a scaling criteria based on data reliability, accuracy, or other parameter.

FIG. 4 includes motion sensor 110C with a coordinate system. Motion sensor 110C can generate an output signal corresponding to motion that can be described as pitch 405 (movement or rotation about the x-axis), roll 410 (about the y-axis), and yaw 415 (about the z-axis).

The relative alignment of an optical sensor (as part of physiological sensor 120A, for example) and an axis of sensitivity of motion sensor 110C can be selected according to a particular application. For example, the optical sensor can be aligned so that a direction of light emission from a light emitting diode (LED) is aligned with a z-axis.

For a limb-worn device having an accelerometer with one axis of sensitivity (z-axis), the LED can be aligned to emit along the z-axis. In this configuration, for example, a toe-worn physiological sensor can be correlated with movement of a leg during flexion and extension of a knee joint. A one axis accelerometer may be suitable for an ambulatory user.

For a limb-worn device having an accelerometer with two axes of sensitivity (x-axis and y-axis), the LED can be aligned to emit along the z-axis. This configuration allows, for example, detection of limb rotation in which the palm is rotated to face up or face down (supination, pronation) and bending of the elbow (flexion, extension). A two axes accelerometer may be suitable for sleep study analysis.

For a limb-worn device having an accelerometer with three axes of sensitivity (x-axis, y-axis, and z-axis), the LED can be aligned to emit along any particular axis. This configuration allows, for example, detection of patient movement such as during transportation in an ambulance or wheel chair.

A particular motion sensor can be configured to detect gross movements of a user. A gross movement relates to use of the large muscles of the human body, such as those in the legs, arms, and abdomen.

### Additional Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown and described. However, the present inventors also contemplate examples in which only those elements shown and described are provided.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, the code may be tangibly stored on one or more volatile or non-volatile computer-readable media during execution or at other times. These computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. A device comprising:
a first sensor having an optical detector and an optical emitter, the optical detector to generate a first output using the optical emitter, the first output corresponding to a physiological parameter of a user;
a motion sensor to generate a motion output corresponding to a detected motion of the user, the motion sensor configured for attachment to the user; and
a processor coupled to the first sensor by a first link and coupled to the motion sensor by a second link, at least one of the first link and the second link includes a wireless communication channel, the processor to generate a processor output using the first output and the motion output.

2. The device of claim 1 wherein the wireless communication channel includes a radio frequency transceiver.

3. The device of claim 1 wherein the first sensor is affixed to the motion sensor by a housing.

4. The device of claim 1 wherein the first sensor is affixed to the processor by a housing.

5. The device of claim 1 wherein the motion sensor is affixed to the processor by a housing.

6. The device of claim 1 wherein the processor is coupled to an interface, the interface configured to communicate with a remote device.

7. The device of claim 1 wherein the processor is coupled to a memory.

8. The device of claim 1 wherein the first sensor includes a pulse oximetry sensor.

9. The device of claim 1 wherein the first sensor is configured for affixation to at least one of a finger of the user, a limb of the user, a head of the user, and a torso of the user.

10. The device of claim 1 wherein the motion sensor includes an accelerometer.

11. A system comprising:
a local unit having a first processor coupled by a first link to a motion sensor and coupled by a second link to a physiological sensor, the motion sensor configured to generate a motion output corresponding to motion of a user and the physiological sensor configured to generate a physiological output corresponding to the user, at least one of the first link and the second link including a wireless communication channel, the first processor coupled to a first interface, and
a remote unit having a second processor coupled to a second interface, the second interface in communication with the first interface and the second processor configured to generate a detector output corresponding to the motion output and the physiological output.

12. The system of claim 11 wherein the second processor is coupled to at least one of a computer, a printer, a database, and a network.

13. The system of claim 11 wherein the second interface and the first interface are coupled by a radio frequency transceiver.

14. The system of claim 11 wherein the motion sensor includes an accelerometer.

15. The system of claim 11 wherein the physiological sensor includes a pulse oximetry sensor.

16. The system of claim 11 wherein the local unit includes a housing, the housing coupled to at least one of the motion sensor and the physiological sensor, the motion sensor and the first processor, and the physiological sensor and the first processor.

17. The system of claim 11 wherein the local unit is configured to be worn by the user.

18. The system of claim 11 wherein the second processor is configured to execute instructions to correlate the first signal and the second signal.

19. The system of claim 11 wherein the remote unit is configured to communicate with at least one of a processor, a printer, a display, and a storage device.

20. An apparatus comprising:
a first sensor coupled to a first housing and configured to generate a first signal corresponding to a physiological parameter of a user;
a second sensor coupled to a second housing, the first housing and the second housing coupled by a physical link, the second housing configured to be worn by a user, the second sensor configured to generate a second signal corresponding to motion of the user; and
a telemetry unit coupled to at least one of the first housing, the second housing, and the physical link, and wherein the telemetry unit is configured for wireless communication of data corresponding to the first signal and the second signal.

21. The apparatus of claim 20 further including a processor coupled to the telemetry unit, the processor configured to execute instructions to correlate the first signal and the second signal.

22. The apparatus of claim 20 wherein the first sensor includes a pulse oximetry sensor.

23. The apparatus of claim 20 wherein the first housing includes at least one of a finger aperture and a limb aperture.

24. The apparatus of claim 20 wherein the physical link includes at least one of a wire conductor and an optical fiber.

25. The apparatus of claim 20 wherein the second sensor includes an accelerometer.

26. The apparatus of claim 20 wherein the telemetry unit includes at least one of a radio frequency (RF) transceiver and an optical transceiver.

27. A method comprising:
generating a first output using an optical emitter and an optical detector, the first output corresponding to a physiological parameter of a user;
generating a motion output using a motion detector, the motion output corresponding to a detected motion of the user, the motion detector configured for attachment to the user;
using at least one wireless communication channel to communicate the first output and the motion output to a processor; and
generating a processor output using a processor executing instructions and using the first output and the motion output.

28. The method of claim 27 wherein generating the processor output includes correlating the first output and the motion output.

29. The method of claim 27 wherein generating the processor output includes compensating the first output using the motion output.

30. A method comprising;
generating a first signal corresponding to a physiological parameter at a first site of a user;
generating a second signal using a user-worn sensor, the second signal corresponding to movement of the user, wherein the first site differs from a location of the user-worn sensor;
using a physical link to couple the first signal and the second signal; and
wirelessly communicating data corresponding to the first signal and the second signal to a remote device.

31. The method of claim 30 further including identifying a relationship as to the first signal and the second signal.

32. The method of claim 30 further including correlating the first signal and the second signal.

33. The method of claim 30 further including compensating the first signal using the second signal.
